(19) United States
(12) Patent Application Publication
(10) Pub. No.: US 2023/0312352 A1
(43) Pub. Date: Oct. 5, 2023

(54) ANTIBACTERIAL HYPER-BRANCHED POLYMER AND ANTIBACTERIAL SPHERICAL ACTIVATED CARBON USING THE SAME

(71) Applicants: KOREA RESEARCH INSTITUTE OF CHEMICAL TECHNOLOGY, Daejeon (KR); NF CO., LTD., Busan (KR)

(72) Inventors: Jeong Kwon SUH, Daejeon (KR); Ji Sook HONG, Daejeon (KR); Sang Kon LEE, Busan (KR); Jae Hoon LEE, Ulsan (KR)

(21) Appl. No.: 18/012,772

(22) PCT Filed: Jun. 30, 2021
(86) PCT No.: PCT/KR2021/008250

§ 371 Date: Dec. 23, 2022

(30) Foreign Application Priority Data
Jul. 16, 2020 (KR) 10-2020-0087877

(51) Int. Cl. C01B 32/318; A01N 59/00

(52) U.S. Cl. CPC C01B 32/318; A01N 59/00; C01P 2004/32; C01P 2004/61; C01P 2006/12; C01P 2006/90

(57) ABSTRACT

The present invention provides a hyper-branched polymer having a three-dimensional structure, which is applicable to treatment of harmful substances in the atmosphere and has antibacterial activity, as well as a method for manufacturing the same. Further, the present invention provides an antibacterial spherical activated carbon which is ultimately obtained using the hyper-branched polymer as a precursor. The antibacterial spherical activated carbon, which is the final product according to the present invention, may induce a chemical bonding of ion bonds in an antibacterial component to a carrier without use of a separate binder, thereby stably immobilizing the antibacterial component. Furthermore, it is possible to adjust a loading amount of the antibacterial component as well as a specific surface area of the carrier through a simple operation. Moreover, the present invention enables uniform distribution of the antibacterial component on the spherical activated carbon at the atomic level, and has an advantage in that a high-strength antibacterial spherical activated carbon can be manufactured without elution and/or loss of antibacterial component even when using the activated carbon of the present invention for water treatment. Furthermore, effects of increasing the antibacterial performance by adsorption of organic matter in pores of the activated carbon that has been developed through incompatibility processing-carbonization-activation processes may also be expected. Therefore, the antibacterial spherical activated carbon prepared by the present invention is applicable to not only the removal of harmful substances such as SOx, NOx, VOC, etc. in the atmosphere but also an air purifier using antibacterial ability of the antibacterial material itself. In particular, the present material can be applied to all air cleaning systems requiring antibacterial functions because a large amount of antibacterial component can be uniformly supported on the spherical activated carbon, and eventually, the material has a perfectly spherical shape with a uniform surface and high strength so as to attain high organic matter removal efficiency.

### TECHNICAL FIELD

The present invention relates to a hyper-branched polymer and activated carbon using the same, and more particularly, to a hyper-branched polymer with proved antibacterial effects and spherical activated carbon using the same.

### BACKGROUND ART

Currently, there are several types of effective substances mainly used for antibacterial purposes, such as silver, copper, iron, zinc and nickel, etc., and these antibacterial materials are being applied to air pollutant purification, water treatment, and antibacterial treatment systems, etc. Copper has long been used as a material exhibiting antibacterial properties in various fields. Materials and products using antibacterial metals such as silver, copper, etc., which are typically proven to have antibacterial properties, have been known for a long time. However, when these materials and products are exposed to a surface in order to express antibacterial performance, they are separated from the surface over a certain period of time and the antibacterial properties disappear immediately. On the other hand, when firmly immobilizing them to a carrier using a binder, or the like, antibacterial components are covered with the binder, etc., and thus cause a problem in that the antibacterial properties cannot be exhibited. In order to solve this problem, various techniques, and the like have been proposed in the art.

Reactions intended to use the antibacterial component may be largely divided into a slurry type reaction in which nano-sized antibacterial components are dispersed and used, and an immobilized type reaction in which the antibacterial component is used while being fixed to a carrier. In the case of the dispersion type reaction, the antibacterial performance is good due to a large surface area of fine particles, however, there are drawbacks in that the system should be supplemented to prevent the separation of the dispersed nano-particles and a complicated follow-up process for separation of nano-sized antibacterial components after use is required.

In order to support the antibacterial component on an immobilized carrier, there are methods for immobilization through application of various methods such as mechanical coating, CVD, impregnation, deposition, plasma coating, chemical coating, and sol-gel coating, etc. to various carriers such as honeycomb, silica ball, glass, and polymer fiber, etc., as well as techniques for production of particles directly using the antibacterial component powder itself.

Among these prior arts, a technique for immobilizing a catalyst on a thin substrate as in Korean Patent Laid-Open Publication No. 2000-0058790 has a limitation in that a reactor should be operated in a fixed bed type. As described above, when the reactor is operated in a fixed form, processing efficiency is significantly lower than that of a dispersed type reactor. However, since the antibacterial spherical activated carbon with a size of about 300 to 450 μm and a specific gravity of 1 to 2 produced by the present invention is used, the reactor can be operated in a fluidized bed type, and it has an advantage in that the activated carbon can be immobilized on a thin plate.

In addition, as in Korean Patent Laid-Open Publication No. 10-2003-0032775, which is a prior art for immobilizing a catalyst on a general carrier, a fixing support is in the form of metal or silica, and has no special function other than a simple role of the fixing support. Further, in the case of silica balls or glass, a photo-reactor is mostly used in the form of a fixed bed in which the carrier does not flow since it is difficult to disperse the support in the photo-reactor due to high density of the carrier. For this reason, there are problems such as inhibition of processing efficiency and increase in operational costs of the reactor.

Korean Patent Laid-Open Publication No. 10-2004-0091385 discloses a magnetic anion exchange resin in which magnetite (Fe3O4) is coated with a cross-linked polymer resin and has an anion exchange functional group introduced on the surface thereof. This can be easily recovered and regenerated, but a particle diameter of the ion exchange resin is as small as 60 to 150 μm, whereas a particle diameter of magnetite is large, thereby entailing a problem in that a sufficient specific surface area for adsorption is not obtained.

### PRIOR ART DOCUMENT

### Patent Document

(Patent Document 1) Korean Patent Laid-Open Publication No. 2000-0058790
(Patent Document 2) Korean Patent Laid-Open Publication No. 10-2003-0032775
(Patent Document 3) Korean Patent Laid-Open Publication No. 10-2004-0091385

### SUMMARY OF INVENTION

### Problems to be Solved by Invention

In consideration of the above-mentioned circumstances, it is an object of the present invention to provide an antibacterial spherical activated carbon, which is produced by mixing an antibacterial component in a manufacturing process of a hyper-branched polymer as a carrier precursor and maintaining the mixture in a spherical shape, in addition, supplementing and maintaining a spherical shape and strength thereof during processing, such that the activated carbon is applicable as a material for treatment of harmful substances in the atmosphere and water treatment, and has even antibacterial activity.

Further, the present invention is intended to solve the use of binder, active ingredient content, a difficulty in regulating a loading intensity, non-uniform distribution of active ingredient, or the like in the carrier, re-detachment of the fixed component from the carrier, etc., which are problems with the existing active ingredient-immobilization techniques such as mechanical coating method, CVD method, impregnation method, deposition method, plasma coating method, chemical coating method, etc.

### Means for Solving Problems

In order to achieve the above objects, the present invention provides a hyper-branched polymer having antibacterial activity, as well as activated carbon using the same.

Further, there are provided a method for production of the hyper-branched polymer and a method for production of the activated carbon.

The hyper-branched polymer can stably immobilize an active ingredient by inducing chemical bonding of the active ingredient to a carrier without use of a separate binder, and may adjust a loading amount of the active ingredient and the specific surface area of the carrier through a simple operation. Further, according to the above hyper-branched polymer, it is possible to uniformly distribute the active ingredient on the carrier at the atomic level, eventually, it has an advantage in that high-strength antibacterial spherical activated carbon can be manufactured without elution or loss of the active ingredients even if it is used for treatment of atmosphere at high-concentration and large flow rate. Moreover, it can also be expected to increase the antibacterial efficiency by the adsorption of viruses, etc. through developed pores of the activated carbon.

The antibacterial spherical activated carbon produced by the present invention may be prepared in the form of a hyper-branched polymer by mixing antibacterial components together in a process of preparing the hyper-branched polymer as a precursor, thereby making it easy to maintain a uniform loading state and loading amount.

Further, there is provided a method for production of an antibacterial spherical activated carbon, including: heat treatment of the hyper-branched polymer through stabilization-carbonization-activation processes thus to convert the polymer into an antibacterial spherical activated carbon with desirably maintained shape and strength, such that the product is applicable to treatment of harmful substance in the atmosphere and water treatment, and further has antibacterial activity.

To achieve the above objects, the present invention may firstly prepare a hyper-branched polymer carrying an antibacterial component, and then produce activated carbon using the same. A shape of the activated carbon is not particularly limited, but is preferably manufactured in a spherical shape.

In order to produce a hyper-branched polymer, it may be prepared using a styrene monomer or polystyrene and divinylbenzene as a crude material. A mixing ratio of the styrene monomer and divinylbenzene is not limited, but preferably, the styrene monomer:divinylbenzene may be included in a weight ratio of 7 to 9.5:0.5 to 3, and more preferably, in a weight ratio of 9:1. If the mixing ratio is out of the above range, a suspension polymerization may not occur.

Ammonium coordination compounds such as Ag, Cu, Fe, Zn, Ni, etc. may be added to the hyper-branched polymer as an antibacterial component, respectively, or as a mixture thereof. The antibacterial component is not limited thereto. The above antibacterial component may be mixed in an amount of 0.001 to 3% by weight (“wt. %”), and preferably 0.01 to 2 wt. % based on the weight of the hyper-branched polymer. If the content is less than the above range, antibacterial effects may not be exhibited. Further, if the content is higher than the above range, there is no significant difference in the antibacterial efficacy, but the polymer may not be well manufactured.

When preparing the hyper-branched polymer, in addition to the antibacterial component, formic acid may be added. The formic acid is not limited but preferably has a concentration of 20 to 40% (v/v). As the formic acid is added, the mixed antibacterial metal may facilitate chemical bonding with the crude materials for production of the hyper-branched polymer, and may induce a uniform distribution.

Thereafter, ultrasonic treatment may be additionally performed for 5 to 60 minutes to form a hyper-branched polymer containing an antibacterial component supported therein by chemical bonding of these mixtures with each other. When treated for less than the time, the antibacterial component may not be supported. On the other hand, when treated for more than the time, it may be difficult to form the polymer.

Further, ultrasound is not limited, but may be in a range of 20 kHz to 25 MHz commonly known in the art.

The hyper-branched polymer may be manufactured as activated carbon through heat treatment. The heat treatment may include stabilization treatment, carbonization treatment, and activation treatment.

The hyper-branched polymer can be stabilized for 2 to 5 hours in a temperature range of 250 to 350° C. by increasing the temperature at 1 to 5° C./min in an atmospheric condition. If the temperature and time are out of the above ranges, oxygen insertion may be insufficient and the spherical shape may be deformed.

The stabilized hyper-branched may be carbonized for 0.5 to 2.0 hours in a temperature range of 500 to 900° C., preferably 700 to 900° C., and more preferably at 700° C. by increasing the temperature at 1 to 3° C./min in a nitrogen atmosphere. If the temperature and time are out of the above ranges, immobilization and carbonization of the carbon component may be insufficient.

In order to form and activate micropores in the formed spherical carbide, it may be activated at 850 to 1,100° C., and preferably at 900° C. for 0.2 to 3 hours in a nitrogen and water vapor atmosphere. If the temperature and time are out of the above ranges, it may be difficult to optimize the pore distribution, and the yield may be reduced.

With regard to the spherical activated carbon according to the present invention, the antibacterial component content in the spherical activated carbon may be adjusted to 0.01 to 20 wt. %, preferably 0.01 to 15 wt. %, and more preferably 0.1 to 12 wt. % in terms of a weight ratio.

When stabilized, carbonized, and activated using the hyper-branched polymer to prepare antibacterial spherical activated carbon, it may exhibit a perfect spherical shape with an even surface, and an average particle diameter may range from 100 to 1,000 μm, preferably 200 to 700 μm, and more preferably 300 to 500 μm.

The strength defined herein refers to a weight that one particle of the prepared antibacterial spherical activated carbon (a unit) can withstand, and all particles are applicable to commercial processes. Further, the strength of the activated carbon according to the present invention may range from 1 to 20 kg/a unit, preferably 3 to 15 kg/a unit, and more preferably 4 to 10 kg/a unit.

The hyper-branched polymer that has undergone the carbonization process according to the present invention may have a specific surface area of 200 to 550 m²/g. Further, the activated carbon using the hyper-branched polymer may have a specific surface area of 800 to 2000 m²/g, and preferably 1,000 to 1,800 m²/g.

A specific gravity of the activated carbon according to the present invention may range from 1 to 3, preferably 1.3 to 2.5, and more preferably 1.4 to 2.

Further, the present invention may provide a method for manufacturing activated carbon which includes a hyper-branched polymer, including:
(a) stabilizing a hyper-branched polymer at 250 to 350° C. for 2 to 5 hours in an atmospheric condition;
(b) carbonizing the hyper-branched polymer stabilized in step (a) at 500 to 900° C., preferably 700 to 900° C. for 0.5 to 2 hours by heating the same while increasing the temperature in a nitrogen atmosphere; and
(c) activating the hyper-branched polymer carbonized in step (b) for 0.2 to 3 hours by heating the same while increasing the temperature to 850 to 1,100° C., preferably to about 900° C. along with nitrogen and water vapor.

The antibacterial activity of the activated carbon according to the present invention is very excellent, therefore, the activated carbon may have antibacterial activity of up to 99.99% based on E. coli. The antibacterial activity preferably provides sterilization effects such as antifungal and cleaning functions (deodorization) as well as antibacterial activity against the living environment bacteria O-157 E. coli, Staphylococcus aureus, Pseudomonas bacteria and the like.

### Advantageous Effects

The antibacterial spherical activated carbon produced according to the present invention is recognized for its excellent antibacterial activity as well as the role of an adsorbent. In particular, since the antibacterial component can undergo chemical bonding when synthesizing the hyper-branched polymer as a precursor of the activated carbon, the activated carbon is possibly utilized as a semi-permanent antibacterial material without separation and elution thereof, and further exhibits excellent organic matter removal efficacy and strength, whereby it is applicable to any process such as atmosphere treatment or water treatment. Moreover, due to excellent sterilization and/or antibacterial activity, the activated carbon can also be applied to a wide range of fields.

Further, the antibacterial spherical activated carbon according to the present invention attains advantages of: increasing the removal efficiency of harmful substances through adsorption of organic matter in the developed pores; excellent antibacterial activity and sterilization performance that are continuously maintained; and easy recovery after use, thereby exhibiting applicable effects to various commercial processes such as air purification, water treatment, etc.

### MODE FOR CARRYING OUT INVENTION

Hereinafter, the present invention will be described in detail by way of examples and experimental examples.

However, the following examples and experimental examples are merely illustrative of the present invention, and the content of the present invention is not limited to the following examples and experimental examples.

### Example 1

In order to prepare a hyper-branched polymer, which is a precursor of activated carbon, first, styrene and divinylbenzene as crude materials were mixed in a weight ratio of 9:1, followed by adding ammonium silver ([Ag(NH₃)₂]⁺ complex) to the mixed solution in an amount to reach a weight ratio of 0.013 wt. %. Thereafter, a 30% (v/v) aqueous solution of formic acid was added while slowly stirring until a volume of the mixed solution becomes doubled. Then, after treatment with ultrasonic waves in a range of 20 kHz to 25 MHz for 5 minutes or more but less than 60 minutes, the product was further stirred for 2 hours to allow a suspension polymerization to be proceeded. After the suspension polymerization was completed, filtration/washing processes were implemented three times or more, whereby no residue remained in the synthesized polymer. The hyper-branched polymer containing the antibacterial component (Ag) was used as a precursor to be converted into activated carbon. This process was performed as follows.

The synthesized hyper-branched polymer was dried at 110° C. for 12 hours, and then stabilized at 300° C. for 5 hours in an atmospheric condition. At this time, a stabilized sample, in which burn off % progressed by about 20%, was obtained. The sample subjected to the stabilization treatment was carbonized by increasing the temperature to 700° C. in a nitrogen atmosphere at 1° C./min in order to convert the same into activated carbon. Then, after 700° C., activation was conducted with water vapor in a nitrogen atmosphere for 0.5 hours to activate pores of the activated carbon. Among the total amount of the obtained antibacterial spherical activated carbon, the total burn off % generated by the heat treatment was 62%.

### Example 2

In order to prepare a hyper-branched polymer, which is a precursor of activated carbon, first, a styrene monomer and divinylbenzene as crude materials were mixed in a weight ratio of 9:1, followed by adding ammonium silver to the mixed solution in an amount to reach a weight ratio of 0.047 wt. %. Thereafter, a 30% (v/v) aqueous solution of formic acid was added while slowly stirring until a volume of the mixed solution becomes doubled. Then, after treatment with ultrasonic waves in a range of 20 kHz to 25 MHz for 5 minutes or more but less than 60 minutes, the product was further stirred for 2 hours to allow a suspension polymerization to be proceeded.

After the suspension polymerization was completed, filtration/washing processes were implemented three times or more, whereby no residue remained in the synthesized polymer. The hyper-branched polymer containing the antibacterial component (Ag) was used as a precursor to be converted into activated carbon. This process was performed as follows.

The synthesized hyper-branched polymer was dried at 110° C. for 12 hours, and then stabilized at 300° C. for 5 hours in an atmospheric condition. The sample subjected to the stabilization treatment was carbonized by increasing the temperature to 700° C. in a nitrogen atmosphere at 1° C./min in order to convert the same into activated carbon. Then, after 700° C., activation was conducted with water vapor in a nitrogen atmosphere for 0.5 hours to activate pores of the activated carbon.

### Example 3

In order to prepare a hyper-branched polymer, which is a precursor of activated carbon, a styrene monomer and divinylbenzene were mixed in a weight ratio of 9:1, followed by adding ammonium silver to the mixed solution in an amount to reach a weight ratio of 0.12 wt. %. Subsequent steps were the same as in Example 2 to prepare activated carbon.

### Example 4

In order to prepare a hyper-branched polymer, which is a precursor of activated carbon, a styrene monomer and divinylbenzene were mixed in a weight ratio of 9:1, followed by adding ammonium silver to the mixed solution in an amount to reach a weight ratio of 0.62 wt. % Subsequent steps were the same as in Example 2 to prepare activated carbon.

### Example 5

In order to prepare a hyper-branched polymer, which is a precursor of activated carbon, a styrene monomer and divinylbenzene were mixed in a weight ratio of 9:1, followed by adding ammonium silver to the mixed solution in an amount to reach a weight ratio of 1.23 wt. % Subsequent steps were the same as in Example 2 to prepare activated carbon.

### Comparative Example 1

In order to prepare a hyper-branched polymer, which is a precursor of activated carbon, a styrene monomer and divinylbenzene were mixed in a weight ratio of 9:1, followed by adding ammonium silver to the mixed solution in an amount to reach a weight ratio of 0.047 wt. % Thereafter, a 30% aqueous solution of formic acid was added while slowly stirring until a volume of the mixed solution becomes doubled. Then, the product was further stirred for 2 hours thus to allow a suspension polymerization to be proceeded. After the suspension polymerization was completed, filtration/washing processes were implemented three times or more, and the product was converted into activated carbon. These processes are substantially the same as those performed in Examples 1 to 5. In order to examine the necessity and effects of the process of ultrasonic treatment, the same method as in Example 2 was performed except that the process of ultrasonic treatment is omitted.

### Experimental Example 1

Assessment of Physical Characteristics of the Activated Carbon According to the Present Invention

In order to evaluate physical characteristics of the antibacterial spherical activated carbon according to the present invention, average particle diameter, specific gravity, strength, specific surface area and silver content were evaluated using Examples 1 to 5 and Comparative Example 1, and results thereof are shown in Table 1 below. EDS analysis was used to measure the silver content. Further, the activated carbon was oxidized at 900° C. for 2 hours in an atmospheric condition and then dissolved in a nitric acid solution to quantify the Ag amount through AA analysis.

TABLE 1
Physical properties of the antibacterial spherical
activated carbons of Examples 1 to 5 and Comparative
Example 1

| | | Specific | | | Average |
|---|---|---|---|---|---|
| | Ag | surface | Strength | | particle |
| | content | area | (kg/a | Specific | diameter |
| Division | (wt. %) | (m²/g) | unit) | gravity | (μm) |
| Example 1 | 0.11 | 1,750 | 9.2 | 1.43 | 350 |
| Example 2 | 0.43 | 1,530 | 7.9 | 1.57 | 367 |
| Example 3 | 1.12 | 1,415 | 5.8 | 1.64 | 386 |
| Example 4 | 5.34 | 1,251 | 5.1 | 1.75 | 403 |
| Example 5 | 10.76 | 1,123 | 4.7 | 1.92 | 415 |
| Comparative | N.D. | — | — | — | 100 |
| Example 1 | | | | | |

As a result of the evaluation, the amount of silver (Ag) supported on the activated carbon of Example 1 was 0.11 wt. %, the spherical activated carbon had an average particle diameter of 350 μm and a spherical shape which is easy to apply to a fluidized bed process, and the surface is also uniform thus to have the conditions for easy adsorption of organic matter. The strength and the specific gravity were found to be 9.2 kg/a unit and 1.43, respectively, which are applicable not only for atmosphere treatment but also for water treatment. Further, the specific surface area was 1,750 m²/g, which is large to sufficiently induce adsorption of organic matter.

The amount of silver supported on the activated carbon of Example 2 was 0.43 wt. %, and the activated carbon had an average particle diameter of 367 μm and a spherical shape. Further, the strength and the specific gravity were 7.9 kg/a unit and 1.57, respectively, and the specific surface area was 1,530 m²/g.

The amount of silver supported on the activated carbon of Example 3 was 1.12 wt. %, and the activated carbon had an average particle diameter of 386 μm and a spherical shape. Further, the strength and the specific gravity were 5.8 kg/a unit and 1.64, respectively, and the specific surface area was 1,415 m²/g.

The amount of silver supported on the activated carbon of Example 4 was 5.34 wt. %, and the spherical activated carbon had an average particle diameter of 403 μm and a spherical shape. Further, the strength and the specific gravity were 5.1 kg/a unit and 1.75, respectively, and the specific surface area was 1,251 m²/g.

The amount of silver supported on the activated carbon of Example 5 was 10.76 wt. %, and the activated carbon had an average particle diameter of 415 μm and a spherical shape. Further, the strength and the specific gravity were 4.7 kg/a unit and 1.92, respectively, and the specific surface area was 1,123 m²/g.

On the other hand, in Comparative Example 1, silver was not detected at all and the average particle diameter was 100 μm or less, indicating that it was difficult to form a hyper-branched polymer.

### Experimental Example 2

In order to assess the antibacterial activity of the antibacterial spherical activated carbon obtained by the present invention, an antibacterial activity test against E. coli was performed on the spherical activated carbon prepared in each of Examples 1 to 5.

Experiments to evaluate the antibacterial activity were implemented by culturing E. coli bacteria. The strain was cultured in LB medium at 35° C. for a culturing period of 24 hours. Such cultured E. coli were diluted 100 times in sterile distilled water to reach a volume of 100 mL, followed by counting the number of E. coli. Then, 400 mg of antibacterial spherical activated carbon was introduced to come into contact with the bacteria in a culture stirrer at 35° C. for 1 hour, followed by counting the number of coliform groups in order to evaluate the antibacterial activity of the antibacterial spherical activated carbon. For measurement of the number of coliform groups, a membrane filtration method was adopted, which is applied to a test for the number of coliform groups defined in Article 10 of the Framework Act on Environmental Policy, and results thereof are shown in Table 2 below.

As a result of the experiment, when the antibacterial spherical activated carbon prepared by the present invention was administered, a reduction rate of the number of coliform groups was shown. In particular, in the case of the samples according to Examples 2 to 4, reduction efficiency, that is, the reduction rate of the number of coliform groups reached 95% or more, thereby demonstrating very excellent antibacterial activity.

TABLE 2
Antimicrobial activity evaluation of antibacterial
spherical activated carbon prepared in Examples 1 to 5

| | E. coli |
|---|---|
| | reduction rate (%) |
| Sample according to Example 1 | 67.73 |
| Sample according to Example 2 | 95.48 |
| Sample according to Example 3 | 99.99 |
| Sample according to Example 4 | 99.99 |
| Sample according to Example 5 | 99.99 |

### Experimental Example 3

In order to assess the separation of antibacterial components from the antibacterial spherical activated carbon obtained by the method according to Examples 1 to 5, each of the samples of Examples 1 to 5 was prepared as a 10 wt. % solution in distilled water, followed by irradiating each solution with ultrasonic waves for 5 minutes. After irradiating and filtering, the filtrate was subjected to investigation whether Ag component was eluted from the filtrate through atomic absorption spectroscopy (AA). Results thereof are shown in Table 3 below.

TABLE 3
Results of antibacterial component elution test for
antibacterial spherical activated carbon
prepared in Examples 1 to 5

| | Ag Conc. (ppm) |
|---|---|
| Sample according to Example 1 | N.D. |
| Sample according to Example 2 | N.D. |
| Sample according to Example 3 | N.D. |
| Sample according to Example 4 | N.D. |
| Sample according to Example 5 | N.D. |

As a result of the experiment, it could be seen that the antibacterial component was not eluted or separated from the activated carbon according to the present invention even in the aqueous solution irradiated with ultrasonic waves.

### Comparative Example 2

In order to compare the antibacterial activity of antibacterial spherical activated carbon, activated carbon was prepared without carrying an antibacterial component therein. In this regard, the same method as in Example 2 was implemented except that an antibacterial component addition process was omitted. Specifically, in order to prepare a hyper-branched polymer, styrene monomer and divinylbenzene were mixed at a weight ratio of 9:1 in terms of wt. %, followed by adding a 30% (v/v) aqueous solution of formic acid while slowly stirring until a volume of the mixed solution became doubled. Then, after treatment with ultrasonic waves for 5 minutes, the product was further stirred for 2 hours to allow a suspension polymerization to be proceeded. After the suspension polymerization was completed, filtration/washing processes were implemented three times or more, and the product was converted into activated carbon. These processes are substantially the same as those performed in Examples 1 to 5.

### Comparative Example 3

In order to examine the effect of using formic acid, activated carbon was prepared in the same manner as in Example 3. However, in order to prepare a hyper-branched polymer, a styrene monomer and divinylbenzene were mixed at a weight ratio of 9:1 in terms of wt. %, followed by adding ammonium silver to the mixed solution in an amount to reach a weight ratio of 0.12 wt. % based on the weight of the mixed solution. Thereafter, distilled water was slowly added until a volume of the mixed solution became doubled. Then, after treatment with ultrasonic waves for 20 minutes, the product was further stirred for 2 hours to allow a suspension polymerization to be proceeded. Subsequent steps were conducted in the same manner as in Examples 1 to 5.

### Experimental Example 4

The spherical activated carbon obtained as described above was subjected to a comparative test for antibacterial activity by the method presented in Experimental Example 1. Results thereof are shown in Table 4 below.

TABLE 4
Comparative results of antibacterial activity of the
samples prepared in Example 2 and Comparative Example 2

| | E. coli reduction |
|---|---|
| | rate (%) |
| Sample according to Example 2 | 95.48 |
| Sample according to Comparative | 31.81 |
| Example 2 | |

As confirmed in Table 4, the antibacterial activity of the sample according to Comparative Example 2, in which no antibacterial component was supported, was only seen as removal by the spherical activated carbon serving as a carrier. That is, it was determined that no antibacterial effect is involved.

### Experimental Example 5

In order to assess effects of addition of formic acid on formation of the hyper-branched polymer after introduction of the antibacterial component in the present invention, the antibacterial component (Ag) contents in Example 3 and Comparative Example 3, respectively, were evaluated. The evaluation method was conducted in the same manner as in Experimental Example 1. As a result of the experiment, the antibacterial component was detected in Example 3, whereas, in Comparative Example 3 in which formic acid was not added during preparation of the hyper-branched polymer, the antibacterial component was not detected at all. Therefore, it could be found that addition of formic acid during polymer formation may play a cross-linking role to lead a more active and smoother chemical bonding when the antibacterial component and the hyper-branched polymer are formed.

TABLE 5
Comparison of Ag content in samples of Example 3
and Comparative Example 3

| | Ag content (%) |
|---|---|
| Sample according to Example 3 | 1.12 |
| Sample according to Comparative | N.D |
| Example 3 | |

1. Activated carbon produced using a hyper-branched polymer containing an antibacterial component as a precursor.

2. The activated carbon according to claim 1, wherein the activated carbon has a spherical shape.

3. The activated carbon according to claim 1, wherein the activated carbon has an average particle diameter of 300 to 500 μm.

4. The activated carbon according to claim 1, wherein the antibacterial component is one or more coordination compounds selected from the group consisting of silver, copper, iron, zinc and nickel.

5. The activated carbon according to claim 1, wherein the antibacterial component is included in an amount of 0.01 to 15 wt. % based on a total weight of the activated carbon.

6. The activated carbon according to claim 1, wherein the hyper-branched polymer contains styrene:divinylbenzene in a weight ratio of 7 to 9:0.5 to 3.

7. The activated carbon according to claim 1, wherein the activated carbon has a specific surface area of 800 m²/g.

8. The activated carbon according to claim 1, wherein the activated carbon has a strength of 4 to 10 kg/unit.

9. The activated carbon according to claim 1, wherein a specific gravity of the activated carbon ranges from 1.4 to 2.

10. The activated carbon according to claim 1, wherein the hyper-branched polymer is prepared by treating the same with ultrasonic waves for 5 to 60 minutes.

11. The activated carbon according to claim 1, wherein the hyper-branched polymer is prepared using 20 to 40% (v/v) of formic acid.

12. The activated carbon according to claim 1, wherein the activated carbon is used for antibacterial treatment or sterilization.

13. A method for manufacturing activated carbon, comprising:
(a) stabilizing a hyper-branched polymer at 250 to 350° C. for 2 to 5 hours in an atmospheric condition;
(b) carbonizing the hyper-branched polymer stabilized in step (a) at 500 to 900° C. for 0.5 to 2 hours by heating the same while increasing the temperature in a nitrogen atmosphere; and
(c) activating the hyper-branched polymer carbonized in step (b) for 0.2 to 3 hours by heating the same while increasing the temperature to 850 to 1,100° C. along with nitrogen and water vapor.

14. The method for manufacturing activated carbon according to claim 13, wherein the hyper-branched polymer of step (a) is prepared to include a styrene monomer:divinylbenzene in a weight ratio of 7 to 9.5:0.5 to 3.

15. The method for manufacturing activated carbon according to claim 13, wherein the hyper-branched polymer of step (a) is prepared to include 0.01 to 15 wt. % of one or more antibacterial components selected from the group consisting of silver, copper, iron, zinc and nickel based on a total weight of activated carbon.

16. The method for manufacturing activated carbon according to claim 13, wherein the hyper-branched polymer of step (a) is prepared using 20 to 40% (v/v) of formic acid.

17. The method for manufacturing activated carbon according to claim 13, wherein the hyper-branched polymer of step (a) is prepared by treating the same with ultrasonic waves in a range of 20 kHz to 25 MHz for 5 to 60 minutes.
